(19) United States
(12) Patent Application Publication
(10) Pub. No.: US 2013/0096458 A1
(43) Pub. Date: Apr. 18, 2013

(54) BIOPSY PUNCH WITH SAFETY SYSTEM AND METHOD OF MAKING AND USING THE SAME

(71) Applicant: STAT MEDICAL DEVICES, INC., N. Miami Beach, FL (US)

(72) Inventor: Steven SCHRAGA, Surfside, FL (US)

(73) Assignee: STAT MEDICAL DEVICES, INC., N. Miami Beach, FL (US)

(21) Appl. No.: 13/649,731

(22) Filed: Oct. 11, 2012

Related U.S. Application Data
(60) Provisional application No. 61/546,330, filed on Oct. 12, 2011.

(51) Int. Cl. A61B 10/00; B23P 11/00

(52) U.S. Cl. 600/567; 29/428

(57) ABSTRACT

The invention provides for a biopsy device comprising a body, a cutting edge, and at least one of; a movable safety cover, a depth adjustment member, and an arrangement for moving the cutting edge relative to a skin engaging surface.

### CROSS-REFERENCE TO RELATED APPLICATIONS

The instant application is a US non-provisional Application that is based on and claims the benefit of U.S. provisional application No. 61/546,330, filed Oct. 12, 2011, the disclosure of which is hereby expressly incorporated by reference thereto in its entirety.

### BACKGROUND OF THE INVENTION

1. Field of the Invention

The invention relates to a biopsy punch which utilizes one or more features shown in the drawings and/or recited in the claims. A method of making and using the biopsy punch is also disclosed.

2. Discussion of Background Information

A biopsy punch is commonly used to obtain a tissue sample. See follow link: http://www.aafp.org/afp/2002/0315/p1155.html. The disclosure of this information is hereby incorporated herein by reference in its entirety as though set forth in full herein. Various types of such devices are shown in the following link: http://www.google.com/search?q=skin+punch+biopsy&hl=en&biw=1157&bih=628&prmd=i vns&tbm=isch&tbo=u&source=univ&sa=X&ei=3s5TTuHNHoXg0QGqnaHNBQ&sqi=2&v ed=0CFIQsAQ. The disclosure of this information is hereby incorporated by reference in its entirety as though set forth in full.

Such devices are also disclosed in U.S. Pat. No. 6,070,593 to CHASE, U.S. Pat. No. 5,827,199 to ALEXANDER, and U.S. Pat. No. 5,123,907 to ROMAINE. The disclosure of each of these documents is hereby incorporated by reference in its entirety as though set forth in full.

An improved device, however, would be safer and/or include a safety system that has at least one of the following advantageous; (1) it would utilize a safety shield that is normally covering a cutting edge of the device; (2) it would utilize a safety shield that automatically covers the cutting edge when not in use; (3) it would provide for depth of cut adjustment; (4) it would include a mechanism or system to prevent re-use; (5) it would be a single-use device; (6) it would be a multi-use device utilizing replaceable cutting edges; (7) it would include a mechanism or system that covers the cutting edge after use to prevent accidental injury; (8) it would provide for a device that can form a continuous cut with a single linear movement or activation; (9) it would provide for a device that can form a continuous cut precisely and automatically when the device is triggered; and (10) it would provide for a device that can form a continuous cut in a more consistent manner and/or without being dependent on skill level.

It is submitted that there is a need for a biopsy punch or device which has one or more of the above-noted advantages and which are lacking in known devices.

### SUMMARY OF THE INVENTION

According to one non-limiting aspect of the invention, there is provided a biopsy device comprising a body, a cutting edge, and at least one of; a safety cover selectively lockable in at least one retracted position, a safety cover selectively movable to at least one retracted position, a safety cover axially retainable in at least one retracted position, a safety cover axially lockable in at least one retracted position, a movable safety cover, a depth adjustment member, and an arrangement for moving the cutting edge relative to a skin engaging surface.

In embodiments, the body may comprise the cutting edge.

In embodiments, the device may utilize the depth adjustment member.

In embodiments, the depth adjustment member may be mounted to the body.

In embodiments, the depth adjustment member may be movably mounted to the body.

In embodiments, the device may utilize the movable safety cover.

In embodiments, the movable safety cover may cover the cutting edge when in an initial position.

In embodiments, the movable safety cover may be movably mounted to a skin contacting end of the body.

In embodiments, the device may utilize the depth adjustment member and the movable safety cover.

In embodiments, movement of the movable safety cover may be limited by the depth adjustment member.

In embodiments, the device may utilize the movable safety cover and at least one spring for biasing the movable safety cover.

In embodiments, the device may further comprise a device for preventing reuse.

In embodiments, the device may further comprise a device movable from an initial position to a position preventing reuse of the device.

In embodiments, the body may comprise an elongated generally cylindrical body.

According to one non-limiting aspect of the invention, there is provided a biopsy device comprising a body, a skin engaging surface, a cutting edge, and at least one of; an arrangement for moving the cutting edge relative to the skin engaging surface, an arrangement for moving the cutting edge at least axially toward the skin engaging surface, an arrangement for causing rotation of at least one of the cutting edge and the body, and an arrangement for allowing at least one of the cutting edge and the body to be coupled to a rotatable portion of a powered device.

In embodiments, the skin engaging surface may be arranged on a cover mounted to the body.

In embodiments, the cutting edge may be arranged on an axially moveable holder at least partially disposed in the body.

In embodiments, the cutting edge may be arranged on replaceable member.

In embodiments, the device may further comprise a trigger system.

In embodiments, the device may further comprise a device for moving the cutting edge to a trigger-set position.

In embodiments, the device may further comprise a depth adjustment system.

In embodiments, the device may be single-use.

In embodiments, the device may be multi-use.

According to one non-limiting aspect of the invention, there is provided a method making the device of anyone of the types disclosed herein, wherein the method comprises arranging a safety shield on a body.

According to one non-limiting aspect of the invention, there is provided a method using the device of anyone of the types disclosed herein, wherein the method comprises placing a safety shield against a tissue surface and forming a cut in the tissue surface with the device.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a side cross-section view of a first non-limiting embodiment of a biopsy device in accordance with the invention;

FIG. 2 shows the biopsy device of FIG. 1 in a specific depth of cut position;

FIG. 3 shows the biopsy device of FIG. 1 with the spring removed and with the safety shield shown prior to installation;

FIG. 4 shows a side cross-section view of another non-limiting embodiment of a biopsy device in accordance with the invention;

FIG. 5 shows the biopsy device of FIG. 4 in a post-use position which prevents re-use;

FIG. 6 shows a side cross-section view of another non-limiting embodiment of a biopsy device in accordance with the invention;

FIG. 7 shows a side cross-section view of another non-limiting embodiment of a biopsy device in accordance with the invention;

FIG. 8 shows a side cross-section view of another non-limiting embodiment of a biopsy device in accordance with the invention;

FIG. 9 shows the biopsy device of FIG. 8 in a post-use position;

FIG. 10 shows a side cross-section view of another non-limiting embodiment of a biopsy device in accordance with the invention;

FIG. 11 shows the biopsy device of FIG. 10 in a post-use position which prevents re-use and completely covers the cutting edge;

FIG. 12 shows a side cross-section view of another non-limiting embodiment of a biopsy device in accordance with the invention. This embodiment utilizes an installable and/or replaceable cutting edge;

FIG. 13 shows the biopsy device of FIG. 12 in a use position;

FIG. 14 shows a top view of the biopsy device of FIG. 12;

FIG. 15 shows a side cross-section view of another non-limiting embodiment of a biopsy device in accordance with the invention. This embodiment utilizes a removable and/or replaceable cutting edge. The cover is shown in an un-installed state;

FIG. 16 shows a side cross-section view of another non-limiting embodiment of a biopsy device in accordance with the invention. This embodiment utilizes a removable and/or replaceable cutting edge and a system for triggering the device;

FIG. 17 shows the biopsy device of FIG. 16 in a triggered position;

FIG. 18 shows a side cross-section view of another non-limiting embodiment of a biopsy device in accordance with the invention. This embodiment utilizes system for adjusting a cutting depth as well as a system for triggering the device;

FIG. 19 shows a side cross-section view of another non-limiting embodiment of a biopsy device in accordance with the invention. This embodiment is similar to that of FIG. 15 except that it is single-use;

FIGS. 20-22 show the biopsy device of FIG. 19 in original, cutting and post cutting positions;

FIG. 23 shows a side cross-section view of another non-limiting embodiment of a biopsy device in accordance with the invention. This embodiment can be used with a portable drill so as to cause rotation of the device during cutting;

FIG. 24 shows a side cross-section view of another non-limiting embodiment of a biopsy device in accordance with the invention. This embodiment is similar to that of FIG. 23 except that an interface member is used to couple the drill to the biopsy device;

FIG. 25 shows a side view of the interface member coupled to a drill chuck;

FIG. 26 shows a bottom view of the interface member shown in FIG. 25;

FIG. 27 shows a side cross-section view of another non-limiting embodiment of a biopsy device in accordance with the invention. In FIG. 27, the safety shield is in a use or retracted position; and

FIG. 28 shows the biopsy device of FIG. 27 with the safety shield retained in further retracted position. This can occur when the user rotates the safety shield relative to the body so that it moves from the position shown in FIG. 27 to that of FIG. 28. In this position, the safety shield can be said to be in any one of; a retained position, a maximum retracted position, a selective retained position, or a locked retracted position.

### DETAILED DESCRIPTION OF THE INVENTION

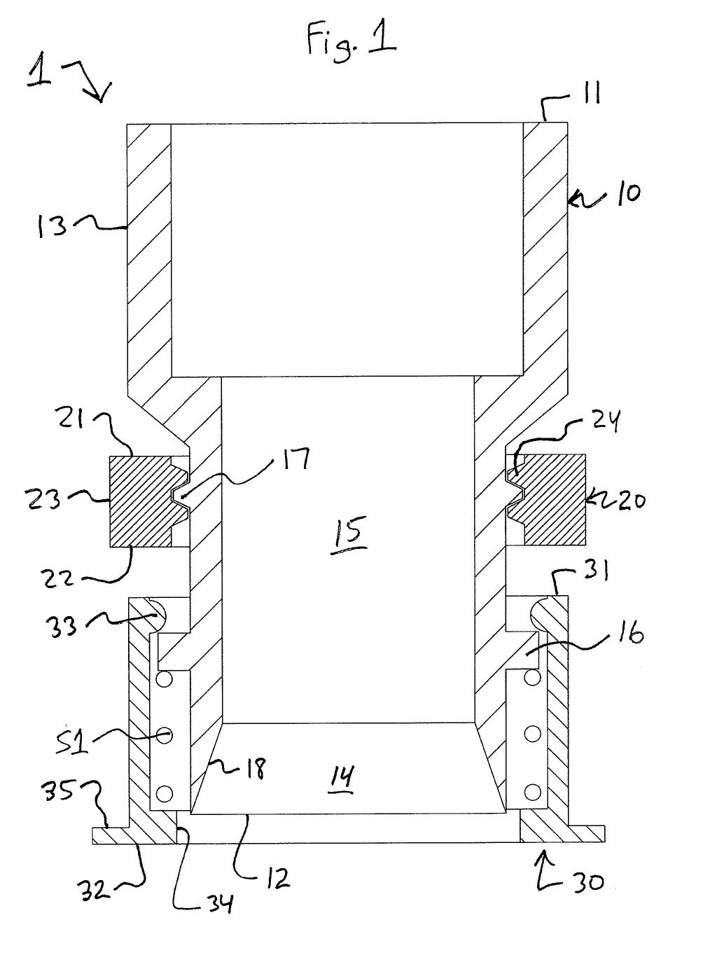

FIGS. 1-3 show a first non-limiting embodiment of the invention. In this embodiment a biopsy device 1 is shown which utilizes three main components and a spring. A first component is a body 10 which has an upper or first end 11 and a lower or second end 12. A gripping portion or section 13 is arranged in an area of the first end 11. A cutting portion or section 14 is arranged in an area of the second end 12. The section 14 is defined by an inner tapered surface 18 having or defining a sharpened circular cutting edge 12. The cutting edge 12 is preferably a very sharp cutting edge so that cutting occurs with as little force as necessary. Such sharpness can meet or exceed known cutting standards used in the art of surgical or biopsy devices without leaving the scope of the invention. The edge 12 can also be serrated or otherwise be made up of interrupted cutting sections. A generally cylindrical internal space 15 is arranged in an area of the body between the cutting portion 14 and the gripping section 13. A circumferential projection 16 is arranged on the body 10 to facilitate the mounting of a safety shield 30.

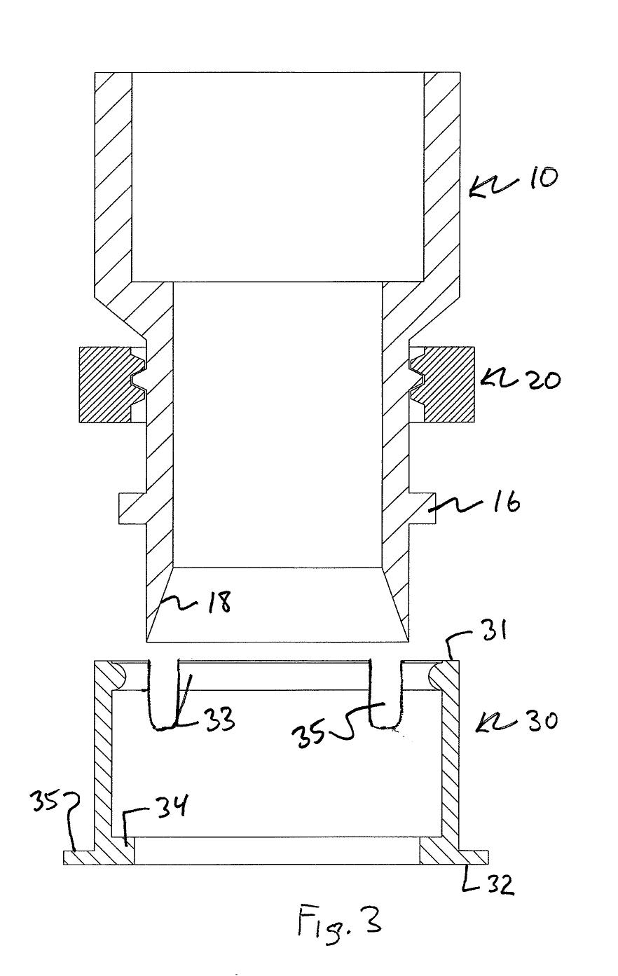

The safety shield 30 has one or more circumferential projections 33 which snap onto or over the projection 16 when installed on the body 10. As can be seen in FIG. 3, the shield 30 as an upper or first end 31, a lower or second open end 32, a generally circumferential internally oriented projection 33 separated by slots 35, and a flange 34. The slots 35 allow the shield 30 to be more easily installed (by allowing for slight circumferential expansion of the portion of the shield 30 having the projection 33) on the body 10. The second end 32 functions as a skin or tissue engaging or contacting surface and is, in embodiments, a generally annular surface.

When mounted to the body 10, and in an original position as shown in FIG. 1, a spring S1 biases the shield 30 toward a covering or protection position, i.e., a position wherein the shield 30 extends out past the cutting edge 14 or sharp end 12. The spring S1 has one or a lower end that contacts an annular surface of the flange 34 of the shield 30 and an upper or other end that contacts an annular surface of the projection 16. In the position shown in FIG. 1, the spring S1 exerts a constant biasing force and causes the projection 33 to contact the projection 16.

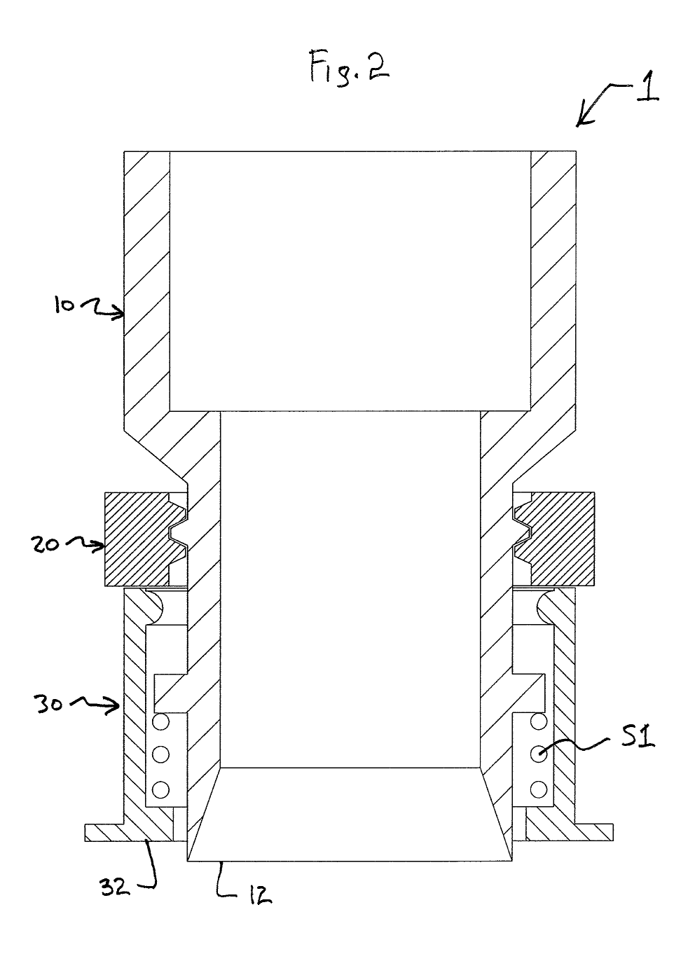

As can be seen in FIG. 2, the safety shield 30 can move axially from the original position shown in FIG. 1 to a retracted position. The amount of the cutting edge 12 or cutting section 14 that passes through the opening in the surface 32 and is exposed (or projects beyond the shield 30) in the retracted position can be regulated by a depth adjustment member 20. The depth adjustment member 20 has an upper generally annular surface 21, a lower generally annular surface 22, an outer generally circumferential grippable surface 23, and an internal thread 24. In embodiments, the surface 23 can be a high friction surface such as, e.g., a knurled surface.

To provide for axial adjustment, the member 20 is connected to the body 10 via a course thread with an internal thread 24 of the member 20 engaging an external thread 17 of the body 10. With such an arrangement, rotation of the member 20 relative to the body 10 (or vice versa) causes the member 20 to move toward or away from the edge 12. Thus, if the member 20 is rotated in one direction, e.g., clockwise, when the shield 30 is moved to the retracted position, it will leave uncovered a different axial amount of the cutting section 14 than when rotated in an opposite direction, e.g., counterclockwise. For example, if the member 20 is rotated so that an axial distance is increased between the edge 20 and the surface 22, when the shield 30 is moved to the retracted position (defined by contact between surface 31 and surface 22), more of the cutting section 14 will be exposed or uncovered by the shield 30. If this occurs when the surface 32 is in contact with skin, a deeper cut will occur in the skin. The depth of the cut will generally be determined by the amount of the cutting edge 12 that extends past the surface 32. On the other hand, if the member 20 is rotated so that an axial distance is decreased between the edge 20 and the surface 22, when the shield 30 is moved to the retracted position (defined by contact between surface 31 and surface 22), less of the cutting section 14 will be exposed or uncovered by the shield 30. If this occurs when the surface 32 is in contact with skin, a shallower cut will occur in the skin. Again, the depth of the cut will generally be determined by the amount of the cutting edge 12 that extends past the surface 32.

In order to use the device shown in FIGS. 1-3, a user can first determine whether the member 20 is position is a desired depth setting. This can occur by arranging an indicator, i.e., an arrow on the body 10, and a series of numbers, i.e., 1-6, or letters on the surface 21 (or optionally surface 23). In case the indicia are numbers, a setting of 3 would be a depth or cut that is between a minimum setting of 1 and a maximum setting of 6. Once in the correct depth setting, the user can grip the device 1 via section 13 with one hand and move the surface 32 into contact with skin or a tissue surface which requires biopsy. The device 1 is then forced into contact with the tissue sufficiently to compress the spring S1 and optionally rotated to cause cutting of the tissue. This occurs until the surface 31 contacts the surface 22 as shown in FIG. 2. At this point, the cutting edge 12 will have reached a maximum predetermined or desired cutting depth as determined by the position of the member 20. Once removed from the tissue, the device 1 will have formed a circular cut in the tissue to a predetermined depth. Next, the biopsy tissue within the circular cut can be removed in the conventional manner.

Although FIGS. 1-3 (as well as other embodiments discussed below) utilize a circular cutting edge 12 (along with a circular body 10 and shield 30), the invention contemplates other cutting edge shapes such as oval, elliptical, polygonal, etc. With other than circular cutting edges, however, cutting would occur via a plunging motion rather than using both a plunging motion and a rotating motion. If the cutting edge 12 is, for example, square shaped, the body 10 and shield 30 can also be square shaped expect that the portion of the body 10 underneath the member 20 should be circular so that the device 1 utilizes depth adjustment via a threaded engagement.

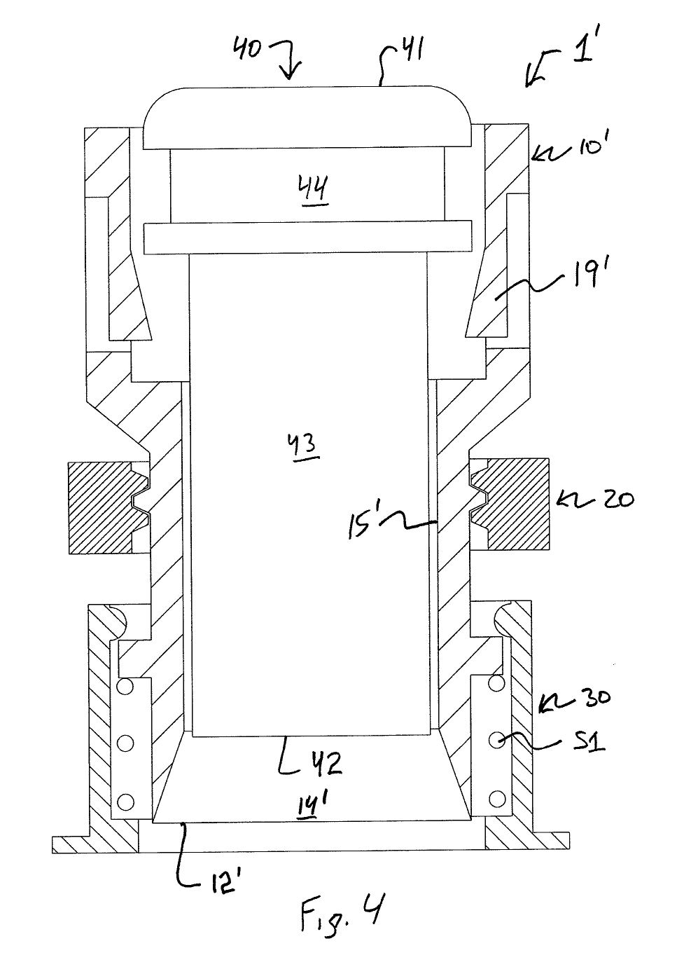

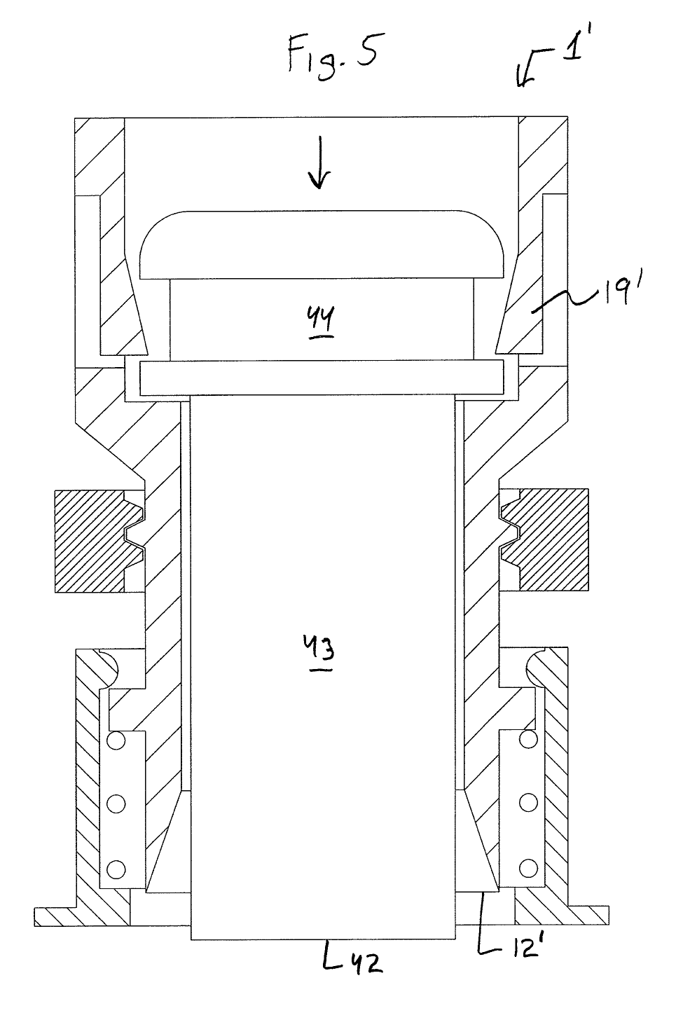

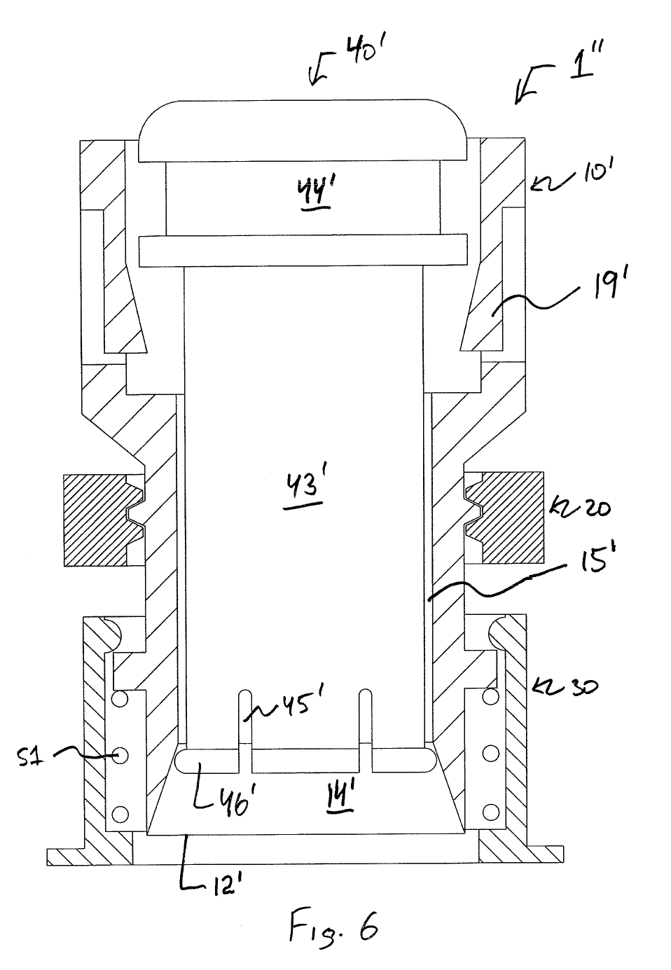

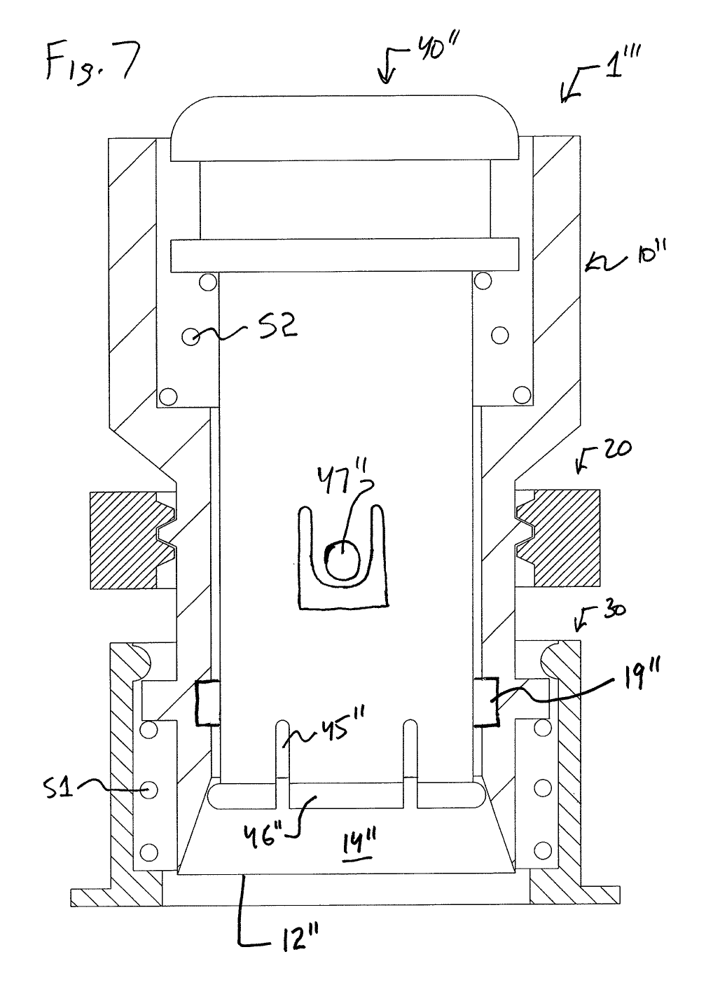

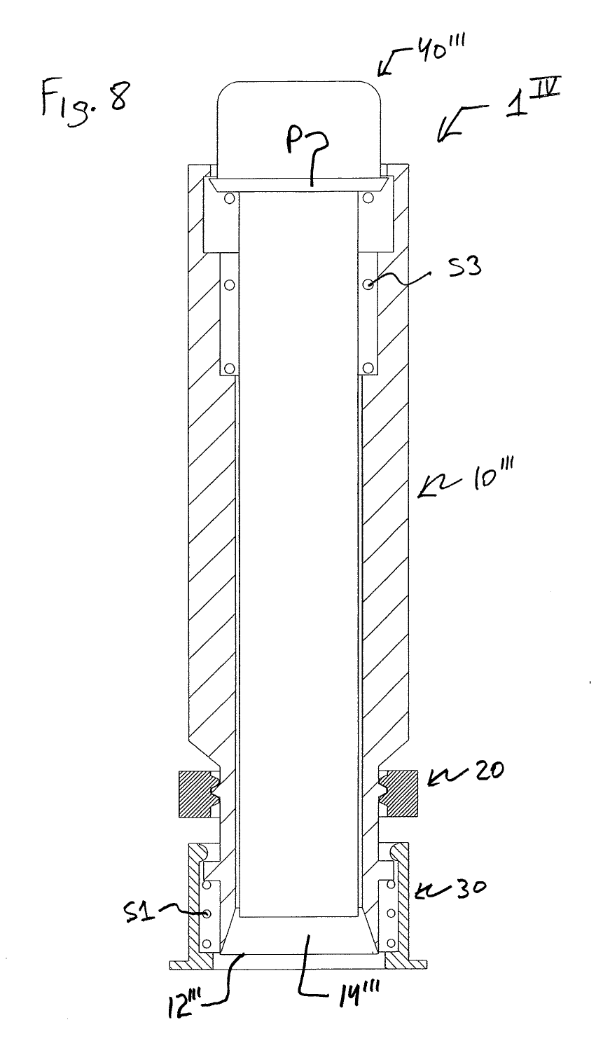

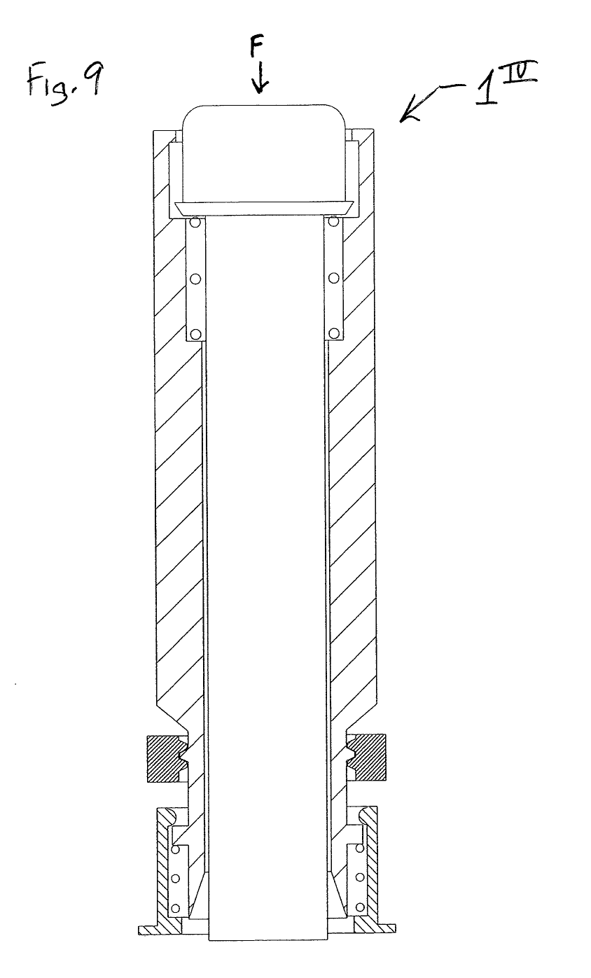

FIGS. 4 and 5 show a second non-limiting embodiment of the invention. In this embodiment the biopsy device 1′ is shown which utilizes four main components and a spring. A first component is a body 10′ which has an upper or first end and a lower or second end as in the previous embodiment. A gripping portion or section is arranged in an area of the first end. In this embodiment, however, one or more deflectable locking projections 19′ are arranged on the grippable section of the body 10′. The deflectable locking projections 19′ can, in embodiments, be equally angularly spaced such that if two are utilized, they are arranged about 180 degrees apart or if four are utilized, they can be arranged about 90 degrees apart. A cutting portion or section 14′ is arranged in an area of the second end. The section 14′ is defined by a tapered surface having a sharpened cutting edge 12′. An internal space 15′is arranged in an area of the body between the cutting portion 14′ and the gripping section. The larger space above the space 15′ allows for inward deflection of the projections 19′. Other main components include the depth adjustment member 20 and the safety shield 30. The fourth main component is a disabling member 40. The member 40 includes an upper end 41, a lower end 42, a main section 43 which can be generally cylindrical, and a locking groove 44.

One purpose of the member 40 is to ensure that the device 1′ cannot be used more than once or one time. Thus, after the device 1′ is used (e.g., in the manner described in the previous embodiment) while the member 40 is in the original position shown in FIG. 4 (and not in contact with tissue), the user can depress or axially move (see arrow in FIG. 5) the member 40 relative to the body 10′ until it reaches the locked position shown in FIG. 5. The locking position is characterized by the projections 19′ becoming non-releasably locked to a groove 44 formed in the member 40. Because of the obstructed or hidden location of the groove 44 and the configuration (or one-way locking feature) of the one or more projections 19′, the member 40 cannot be (or is prevented from being) moved back to the original position after it is locked. The result of locking is that the member 40 is completely and/or permanently axially retained in the locked position. The device 1′ shown in FIG. 5 can thus be rendered a single-use device, i.e., it is disposable after a single-use and/or it can be used once or one time and can then be safely disposed of. An additional advantage or purpose of the member 40, however, relates to the fact that the lower end 42 of the member 40 extends out past the edge 12′ thereby substantially preventing a user from accidentally being cut by the used cutting edge after the member becomes locked. Accidental cutting by the edge 12′ can thus essentially be prevented even if the user mistakenly moves or attempts to move the shield 30 toward the retracted position and against the biasing force of the spring S1. To render the device 1′ even safer, the tapered cutting section 14′ can be reversed so that the generally cylindrical surface defining the inner space 15′ extends to the cutting end or edge 12′ and so that the taper is a circumferential outer taper instead of a circumferential inner taper as shown in FIG. 4. Such a modification can also be utilized in the embodiment of FIGS. 1-3 as well as other embodiments discuss below.

FIG. 6 shows another non-limiting embodiment of the invention. In this embodiment the biopsy device 1″ is shown which utilizes four main components and a spring. A first component is a body 10′ which has an upper or first end and a lower or second end as in the previous embodiment. A gripping portion or section is arranged in an area of the first end. In this embodiment as well, one or more deflectable locking projections 19′ are arranged on the grippable section of the body 10′. The deflectable locking projections 19′ can, in embodiments, be equally angularly spaced such that if two are utilized, they are arranged about 180 degrees apart or if four are utilized, they can be arranged about 90 degrees apart. A cutting portion or section 14′ is arranged in an area of the second end. The section 14′ is defined by a tapered surface having a sharpened cutting edge 12′. An internal space 15′is arranged in an area of the body between the cutting portion 14′ and the gripping section. The larger space above the space 15′ allows for inward deflection of the projections 19′. Other main components include the depth adjustment member 20 and the safety shield 30. The fourth main component is a disabling member 40′. The member 40′ includes an upper end, a lower end, a main section 43′ which can be generally cylindrical, and a locking groove 44′. A circumferential outwardly oriented projection 46′ is arranged in the lower end—as are plural slots 45′. With such an arrangement, once the member 40′ is inserted into the body 10′, it is prevented from falling out of the body 10′. This is because upper axial movement (prior to it being locked to the body) is restrained or limited by contact between the projection 46′ and the surface 14′ and lower axial movement is restrained or limited by contact between the projection arranged below the groove 44′ and the projections 19′. The device 1″ otherwise functions in substantially the same way as that of FIGS. 4 and 5.

FIG. 7 shows another non-limiting embodiment of the invention. In this embodiment the biopsy device 1′″ is shown which utilizes four main components and two springs. The first spring S1 is similar to that described in previous embodiments. However, this embodiment also utilizes a second spring S2 to bias the member 40″ towards an initial or retracted position and away from the locked position. A first component is a body 10″ which has an upper or first end and a lower or second end as in the previous embodiment, which is like that of FIGS. 1-3 in not requiring deflectable projections. Instead, one or more deflectable locking projections 47″ are arranged on a member 40″. The one or more deflectable projections 47″ can deflect radially outwardly when moved into alignment with a circumferential groove 19″ formed in the body 10″. A gripping portion or section is arranged in an area of the first end. In this embodiment, one or more deflectable locking projections can, but need not be arranged on the grippable section of the body 10″. A cutting portion or section 14″ is arranged in an area of the second end. The section 14″ is defined by a tapered surface having a sharpened cutting edge 12″. An internal space is arranged in an area of the body between the cutting portion 14″ and the gripping section. The larger space above the space allows for the second spring S2. Other main components include the depth adjustment member 20 and the safety shield 30. The fourth main component is the disabling member 40″. The member 40″ includes an upper end, a lower end, a main section which can be generally cylindrical, and an optional locking groove at the upper end. Such a groove can be advantageous if one wished to combine the locking feature of FIG. 7, i.e., elements 47″ and 19″, with the projections 19′ used in the previous embodiment in order to provide, e.g., for a dual locking system. A circumferential outwardly oriented projection 46″ is arranged in the lower end—as are plural slots 45″. With such an arrangement, once the member 40″ is inserted into the body 10″, it is prevented from falling out of the body 10″. This is because upper axial movement (prior to it being locked to the body) is restrained or limited by contact between the projection 46″ and the surface 14″ and lower axial movement is restrained by the spring S2 at least until the spring force is overcome. The device 1′″ otherwise functions in substantially the same way as that of FIG. 6.

FIGS. 8 and 9 show another non-limiting embodiment of the invention. In this embodiment the biopsy device 1^{IV} is shown which utilizes four main components and two springs. The first spring S1 is similar to that described in previous embodiments. Like the previous embodiment, a second spring S3 is used to bias the member 40′″ towards an initial or retracted position. A first component is a body 10′″ which is elongated relative to previous embodiments and has an upper or first end and a lower or second end as in the previous embodiment, which is like that of FIGS. 1-3 in not requiring deflectable projections. A gripping portion or section is arranged in an area of the first end. In this embodiment, one or more deflectable locking projections can, but need not be arranged on the grippable section of the body 10′″. A cutting portion or section 14′″ is arranged in an area of the second end. The section 14′″ is defined by a tapered surface having a sharpened cutting edge 12′″. An internal space is arranged in an area of the body between the cutting portion 14′″ and the gripping section. The larger space above the space allows for the second spring S3. Other main components include the depth adjustment member 20 and the safety shield 30. The fourth main component is the disabling member 40′″. The member 40′″ includes an upper end, a lower end, a main section which is elongated and can be generally cylindrical, and a circumferential outwardly oriented projection P is arranged in the upper end. With such an arrangement, once the member 40′″ is inserted into the body 10′″, it is prevented from falling out of the body 10′″. This is because upper axial movement is restrained or limited by contact between the projection P and a shoulder at the upper end of the body 10′″ and lower axial movement is restrained by the spring S3 at least until the spring force is overcome by a force F as shown in FIG. 9. The device 1^{IV} otherwise functions in substantially the same way as that of FIG. 1.

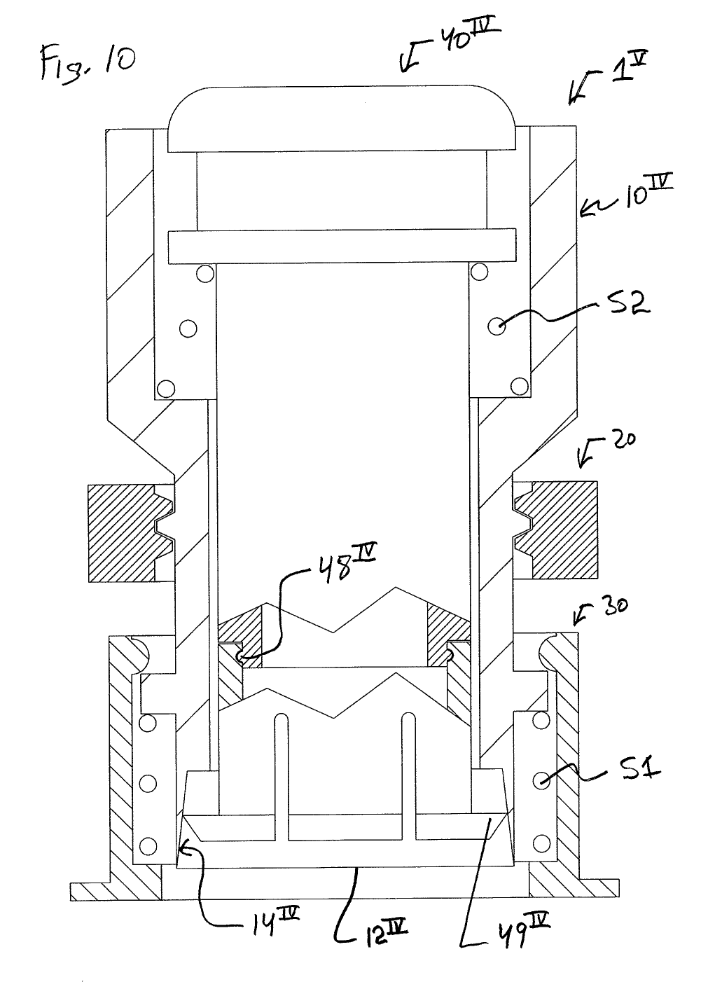

FIGS. 10 and 11 show another non-limiting embodiment of the invention. In this embodiment the biopsy device 1^{V} is shown which utilizes four main components and two springs. The first spring S1 is similar to that described in previous embodiments. Like the previous embodiment, a second spring S2 is used to bias the member 40^{IV} towards an initial or retracted position. A first component is a body 10^{IV} which has an upper or first end and a lower or second end as in previous embodiments and which is like that of FIGS. 1-3 in not requiring deflectable projections. A gripping portion or section is arranged in an area of the first end. In this embodiment, one or more deflectable locking projections can, but need not be arranged on the grippable section of the body 10^{IV}. A cutting portion or section 14^{IV} is arranged in an area of the second end. The section 14^{IV} is defined by a tapered surface having a sharpened cutting edge 12^{IV}. An internal space is arranged in an area of the body between the cutting portion 14^{IV} and the gripping section. The larger space above the space allows for the second spring S2. Other main components include the depth adjustment member 20 and the safety shield 30. The fourth main component is the disabling member 40^{IV}. The member 40^{IV} includes an upper end, a lower end, a main section which includes a connecting interface and can be generally cylindrical. The connection can utilize a circumferential projection 48^{IV} engaging with a circumferential recess. A circumferential outwardly oriented tapered projection 49^{IV} is arranged in lower end. With such an arrangement, once the member 40^{IV} is inserted into the body 10^{IV}, it is prevented from being removed from the body 10^{IV}. This is because upper axial movement is restrained or limited by contact between the projection 48^{IV} and a shoulder at the upper end of the section 14^{IV} and lower axial movement is restrained by the spring S2 at least until the spring force is overcome by a force F as shown in FIG. 11. The device 1^{IV} otherwise functions in substantially the same way as that of FIG. 1 except that when the projection 49^{IV} extends out past the edge 12^{IV} as shown in FIG. 11, the projection 49^{IV} expands circumferentially outwardly to extend over and cover the cutting edge 12^{IV}. This also functions as a locking feature in that it non-releasably locks the member 40^{IV} in the extended position. The biopsy device 1^{V} is thus rendered single-use.

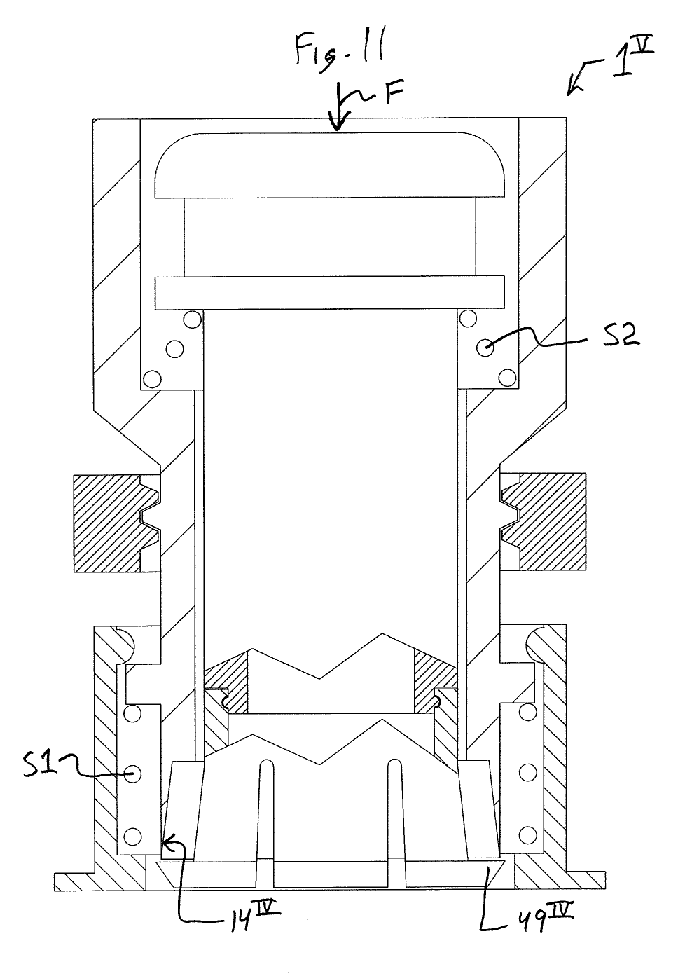

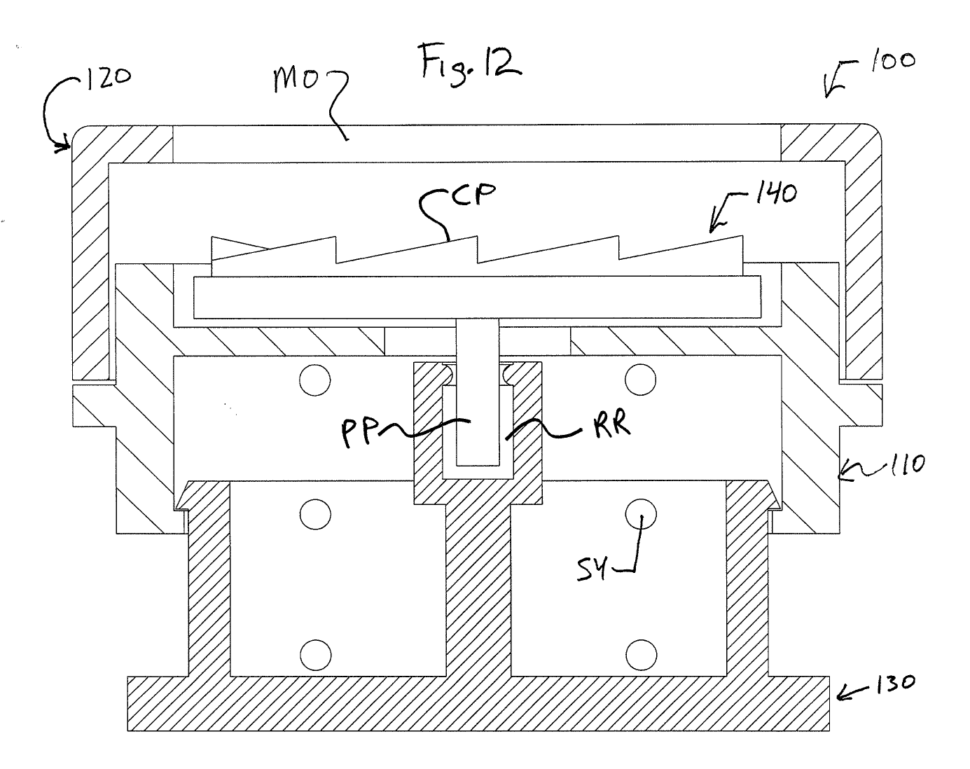

FIGS. 12-14 show another non-limiting embodiment of the invention. In this embodiment the biopsy device 100 is shown which utilizes four main components and one spring. The spring S4 is used to bias the member 130 towards an initial or retracted position relative to the body 110. A first component is the body 110 which has an open upper or first end and an open lower or second end. A gripping portion or section is arranged in an area between the first and second ends. A cutting portion or section CP is arranged on an upper side of the member 140 which forms another of the four components. Other main components include a safety cover 120 and the member 140 which can be configured to be removably mounted to the member 130 via a connection formed by a projecting portion PP and a receiving recess RR. With such an arrangement, one can remove the cap 120 from the body 110 and install or replace the member 140. The device 100 can be used by placing the cap 120 against a tissue surface and depressing the member 140 until the cutting portion CP passes through the main opening MO in the cover 120 and cuts into the tissue. The depth of cutting is determined by the amount of the cutting portion CP that extends put past the tissue engaging surface of the cap 120. Although not shown, the device 100 can also include depth adjustment by providing adjustment (e.g., a threaded connection) between the projection PP and the recess RR (not shown). The biopsy device 100 is thus rendered multi-use except that each time it is used, the member 140 can be replaced with a new member 140.

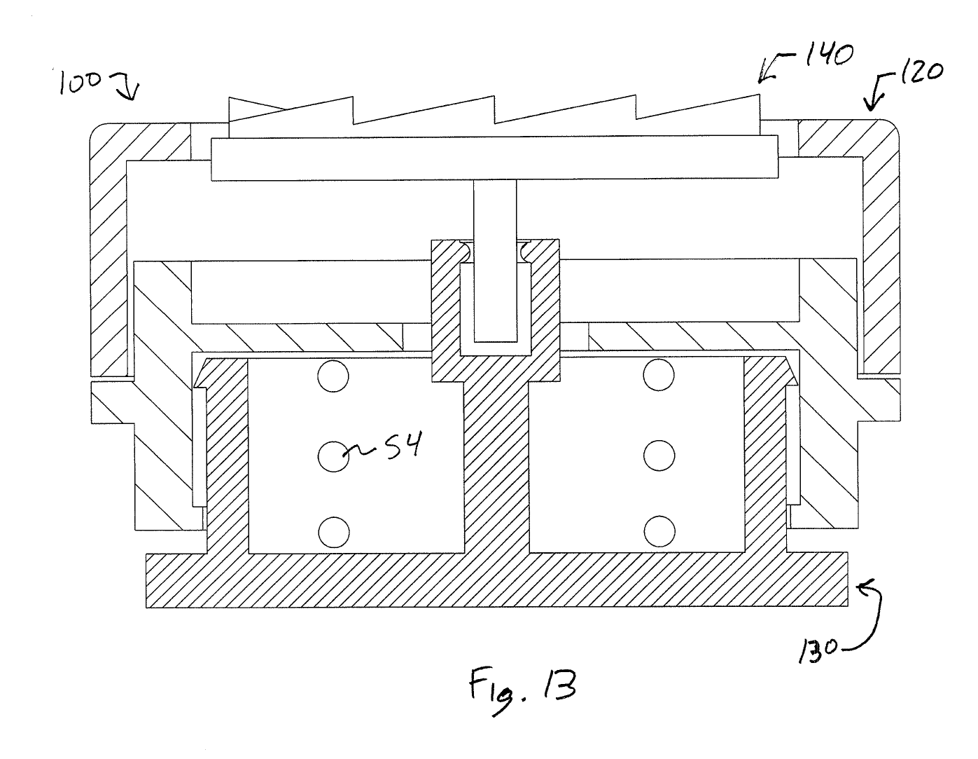

FIG. 15 shows another non-limiting embodiment of the invention. In this embodiment the biopsy device 100′ is shown which utilizes four main components as well as a trigger or moving system. The moving system (not shown) is used to move the member 140′ towards a cutting position from an initial or retracted position relative to the body 110′. A first component is the body 110′ which has an open upper or first end and can be part of a device such as a lancet device (not shown). A gripping portion or section is arranged in an area adjacent the open upper end. A cutting portion or section CP′ is arranged on an upper side of the member 140′ which forms another of the four components. Other main components include a safety cover 120′ and the member 140′ which can be configured to be removably mounted to a holding member HM via a connection formed by a projecting portion PP′ and a receiving recess RR′. With such an arrangement, one can remove the cap 120′ from the body 110′ and install or replace the member 140′. The device 100′ can be used by placing the cap 120′ against a tissue surface and causing the member HM to move until the cutting portion CP′ cuts into the tissue. The depth of cutting is determined by contact between movable stop surface MSS contacting the fixed stop surface FSS. Such contact thus regulates the amount of the cutting portion CP′ that extends put past the tissue engaging surface of the cap 120′. Although not shown, the device 100′ can also include depth adjustment by providing adjustment (e.g., a threaded connection) between the projection PP′ and the recess RR′ (not shown). The biopsy device 100′ is thus rendered multi-use except that each time it is used, the member 140′ can be replaced with a new member 140′. In this embodiment, the member HM can also be made to rotate when in the extended position. Such rotation can occur mechanically or via an electric motor (not shown).

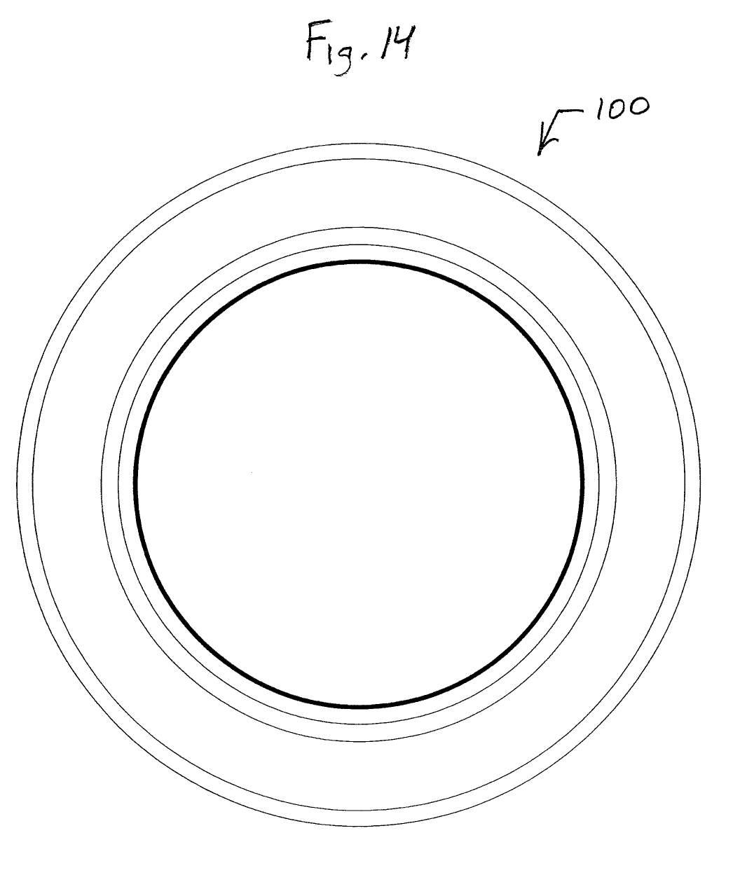

FIGS. 16 and 17 show another non-limiting embodiment of the invention. In this embodiment the biopsy device 100″ is shown which utilizes four main components as well as a trigger or moving system. The moving system is a manual grip 160 used to move the member 130″ towards a cocked or trigger-set position (shown in FIG. 16) from an initial position relative to a two component body 110″a/110″b whose components are fixed to one another. The first component is the body which has an open upper or first end that is covered by a removable cap 120″ fanning another component. A gripping portion or section of the body is arranged in an area adjacent the trigger buttons 170. A cutting portion or section CP is arranged on an upper side of the member 140″ which forms another of the four components. Another main component includes the member 140″ which can be configured to be removably mounted to a holding member portion of the member 130″ via a friction connection. With such an arrangement, one can remove the cap 120″ from the body and install or replace the member 140″. The device 100″ can be used by moving the member 130″ to the cocked position shown in FIG. 16 which compresses the spring S4. Next, the cap 120″ is placed against a tissue surface and the triggers 170 are squeezed against the biasing force of a compressible ring CR (functioning as a biasing member) which causes the member 130″ to move under the axial expansion of the spring S4 until the cutting portion of the member 140″ passes through the opening of the cap 120″ and cuts into the tissue. The depth of cutting is determined by contact between movable stop surface MSS contacting the fixed stop surface FSS. Such contact thus regulates the amount of the cutting portion that extends put past the tissue engaging surface of the cap 120″. Although not shown, the spring S4 can have both axial ends fixed or coupled respectively to the body and member 130″ so that the spring S4 also moves the member 130″ back away from the cutting position shown in FIG. 17 to an original or intermediate position between the positions of FIGS. 16 and 17. The biopsy device 100″ is thus rendered multi-use except that each time it is utilized, the member 140″ can be replaced with a new member 140″. In this embodiment, the member 160 (and also member 130″ and 140″) can also be made to rotate when in the extended position (FIG. 17). Such rotation can occur mechanically by the user. Although not shown, this embodiment can also utilize depth of cutting adjustment by, e.g., using a threaded engagement between the cap 120″ and the body 110″a/110″b.

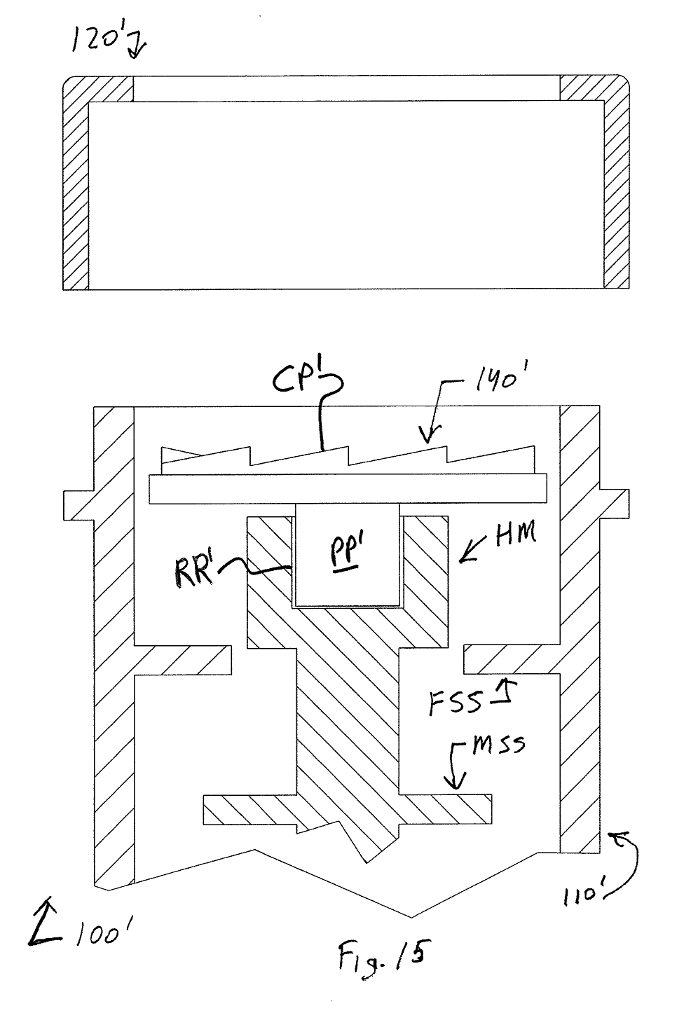

FIG. 18 shows another non-limiting embodiment of the invention. In this embodiment the biopsy device 100′″ can utilize the components shown in the embodiment of FIG. 15 and additionally utilizes a trigger button 160′ that cause the holding member to move to the cutting position. Additionally, a depth adjustment dial or disk 180 allows the user to set a depth of cutting depth as desired by rotating the disk 180 relative to an indicator (i.e., an arrow) arranged on the body. The cap 120′″ can also utilize an alignment mechanism that includes one or more releasable locking projections RLP that engage with one of more recesses RLR.

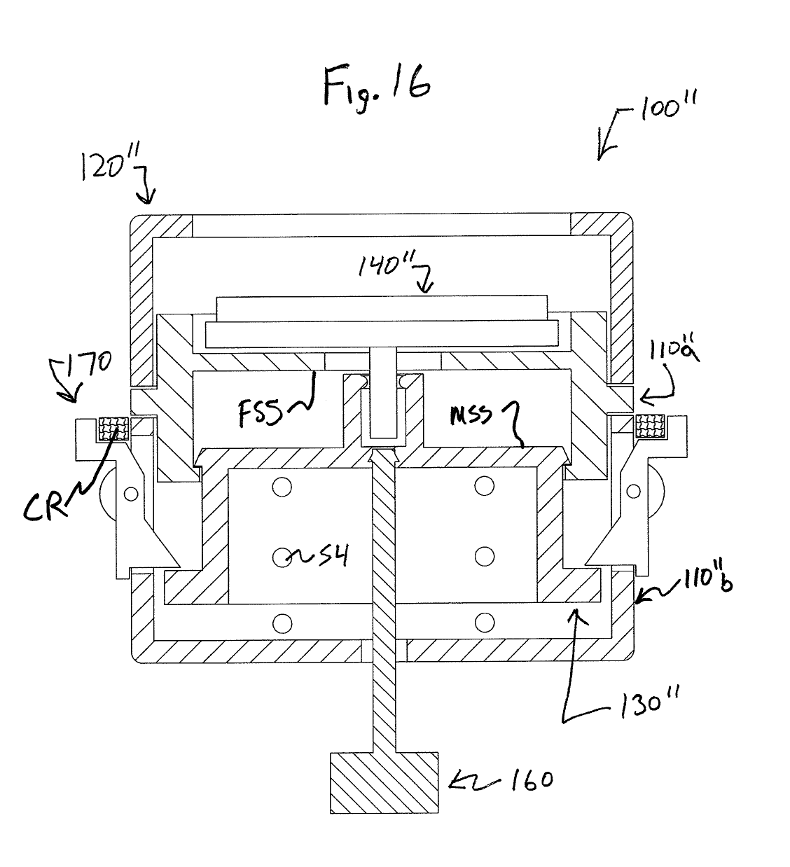

FIG. 19 shows another non-limiting embodiment of the invention. In this embodiment the biopsy device 100^{IV} can utilize the components shown in the embodiment of FIG. 15 and additionally utilizes a non-removable cap 120^{IV}. A non-releasable locking mechanism includes one or more non-releasable locking projections LP that engage with one of more locking recesses LR. With such an arrangement, the device 100^{IV} can be rendered single-use.

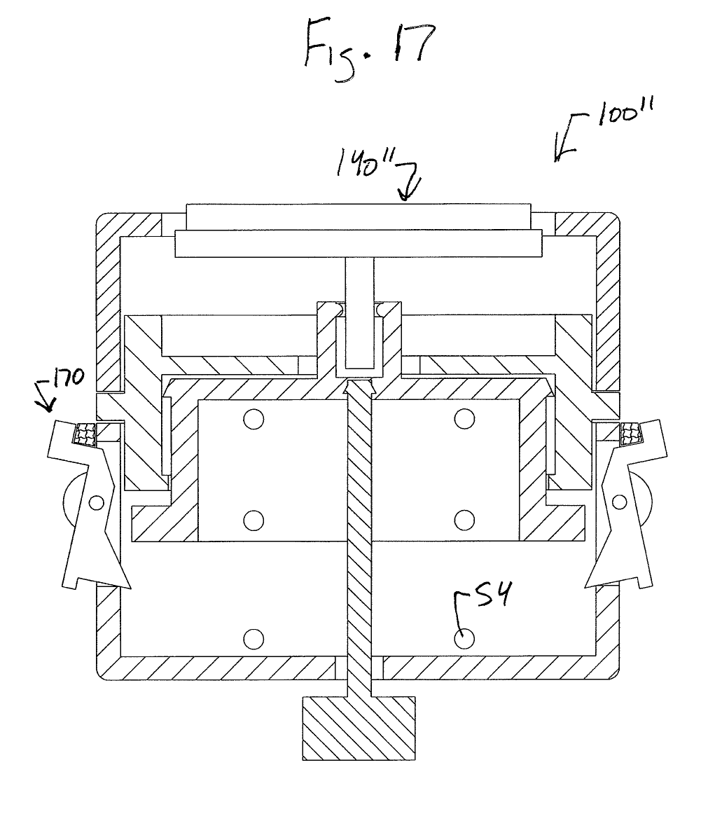

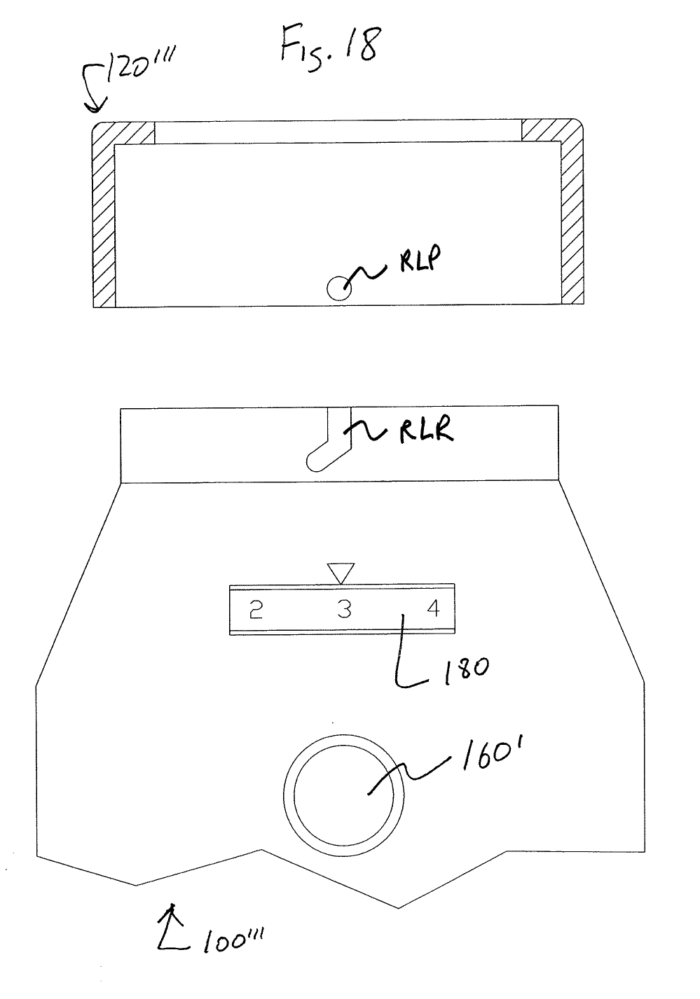

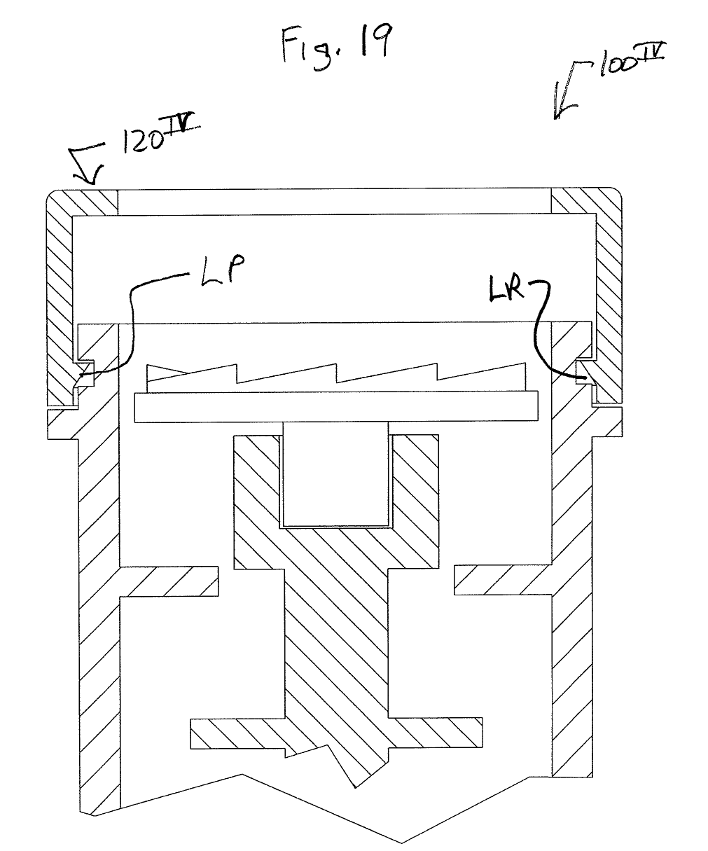

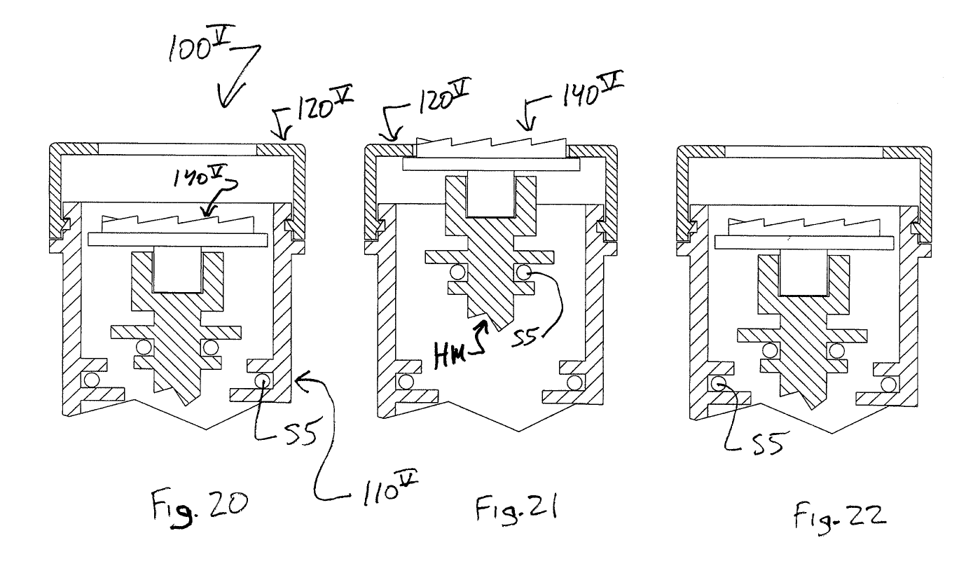

FIGS. 20-22 show another non-limiting embodiment of the invention. In this embodiment the biopsy device 100^{V} can utilize the components shown in the embodiment of FIG. 19 and additionally utilizes a spring S5 having opposite axial ends fixed (or axially retained) to respective portions of the body 110^{V} and the holding member HM. With such an arrangement, the holding member HM can move between a retracted or armed position shown in FIG. 20, to the extended cutting position (with the cutting portion of the member 140^{V} extending out past an outer surface of the cap 120^{V}) and back to either an intermediate position or the armed position shown in FIG. 22. With such an arrangement, the device 100^{V} can also optionally be rendered single-use.

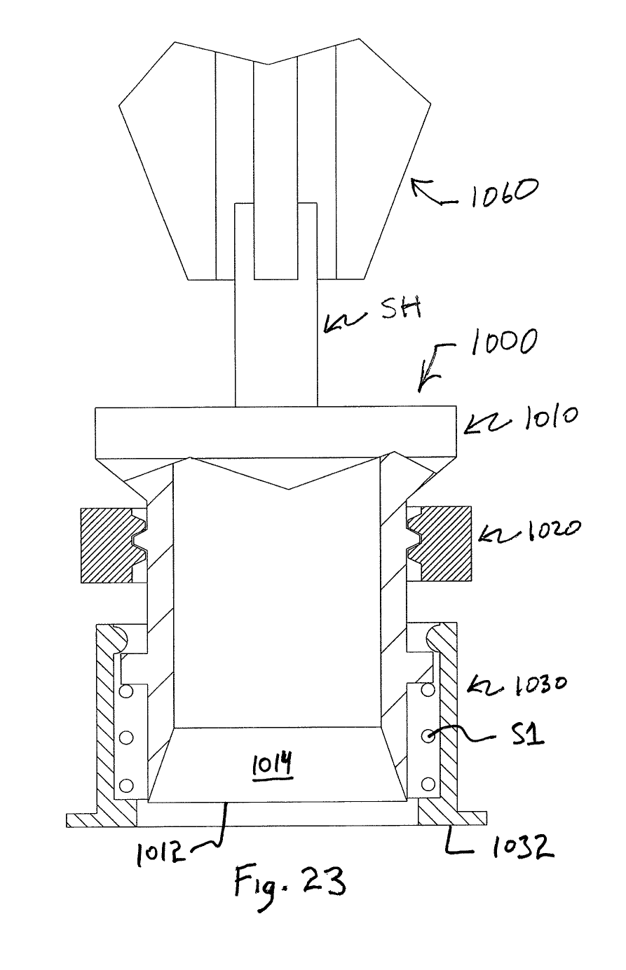

FIG. 23 shows another non-limiting embodiment of the invention. In this embodiment a biopsy device 1000 is shown which utilizes three main components and a spring. A first component is a body 1010 which has an upper or first end and a lower or second end. A tool mounting portion or section SH is arranged in an area of the first end. A tool having a chuck 1060 can be coupled to the section SH in order to cause the body 1010 to rotate. A cutting portion or section 1014 is arranged in an area of the second end. The section 1014 is defined by an inner tapered surface having or defining a sharpened circular cutting edge 1012. The cutting edge is preferably a very sharp cutting edge so that cutting occurs with as little force as necessary.

The safety shield 1030 has one or more circumferential projections which snap onto or over the projection when installed on the body 1010. A spring S1 biases the shield 1030 toward a covering or protection position, i.e., a position wherein the shield 30 extends out past the cutting edge 1014 or sharp end 1012. The spring S1 has one or a lower end that contacts an annular surface of the flange of the shield 1030 and an upper or other end that contacts an annular surface of the projection. In the position shown in FIG. 23, the spring S1 exerts a constant biasing force and causes the projection of the shield 1030 to contact the projection of the body 1010. To provide for axial adjustment, a member 1020 is connected to the body 1010 via a course thread with an internal thread of the member 1020 engaging an external thread of the body 1010.

In order to use the device shown in FIG. 23, a user can first determine whether the member 1020 is position is a desired depth setting. This can occur by arranging an indicator, i.e., an arrow on the body 1010, and a series of numbers, i.e., 1-6, or letters on an outer surface. In case the indicia are numbers, a setting of 3 would be a depth or cut that is between a minimum setting of 1 and a maximum setting of 6. Once in the correct depth setting, the user can insert end SH into a rotatable chuck 1060 of a rotating tool and then move the surface 1032 into contact with skin or a tissue surface which requires biopsy while the device 1000 is caused to rotate. The device 1000 is then forced into contact with the tissue sufficiently to compress the spring S1 with rotation being used to cause cutting of the tissue. This occurs until the rear annular surface of the shield 1030 contacts the front annular surface of the member 1020. At this point, the cutting edge 1012 will have reached a maximum predetermined or desired cutting depth as determined by the position of the member 1020. Once removed from the tissue, the device 1000 will have formed a circular cut in the tissue to a predetermined depth. Next, the biopsy tissue within the circular cut can be removed in the conventional manner.

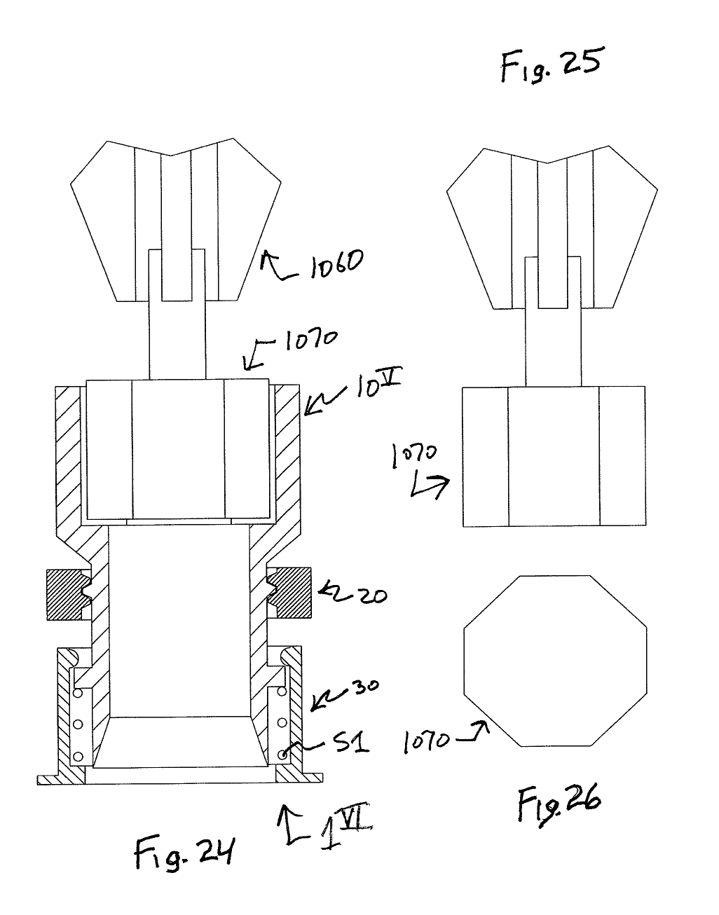

FIGS. 24-26 shows another non-limiting embodiment of the invention. In this embodiment a biopsy device 1^{IV} is shown which utilizes three main components and a spring S1 and which is utilized with a tool and a device 1070 for coupling the device 10^{V} to the tool. A first component is a body 10^{V} which has an upper or first end and a lower or second end. The device 1^{VI} also includes a depth adjuster 20 and a shield 30. A coupling tool mounting recess is arranged at the first end of the body 10^{V} is shaped, e.g., hex shaped, to receive the shaped portion, e.g., hex shaped portion as shown in FIG. 26, of the coupling device 1070. A tool having a chuck 1060 can be coupled to the device 1070. The device 1070 can then be slid into the upper recess of the body 10^{V} to cause it to rotate. The device 1^{VI} otherwise functions like that of FIG. 23.

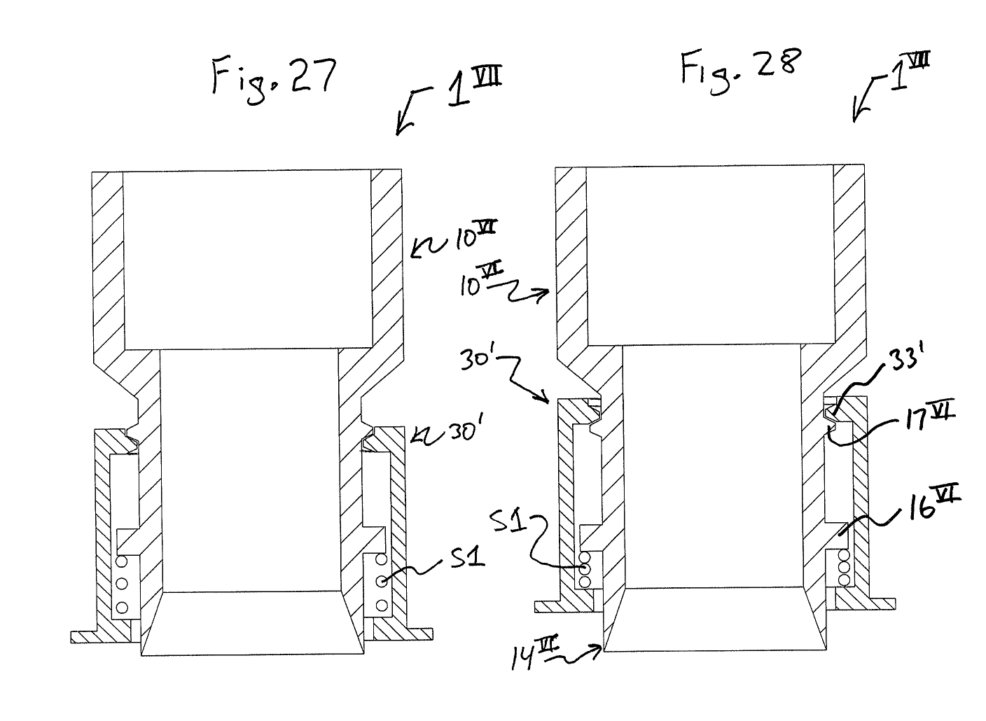

FIGS. 27 and 28 show another non-limiting embodiment of the invention. In this embodiment a biopsy device 1^{VII} is shown which utilizes two main components and a spring S1. These components include a body 10^{VI} and a shield 30′. In order to use the device shown in FIGS. 27 and 28, a user can take the device 1^{VII} in the state shown in FIG. 27 and use it to cut tissue. FIG. 27 shows the shield 30′ in the retracted position as would occur during cutting. After use, the shield 30′ would of course move back via the spring S1 to the original position covering the cutting edge 14^{VI} (not shown, but similar to that of FIG. 1). In addition, should the user desire the retain or lock the shield 30′ in a retracted position, she can move the same to the retracted position shown in FIG. 27 and then rotate the shield 30′ relative to the body 10^{VI} so that the internal thread 33′ of the shield 30′ engages with the external thread 17^{VI} of the body 10^{VI} by an amount sufficient lock or axially retain the shield 30′ in the position shown in FIG. 28. Should the user then desire to place the shield 30′ into a working position again, she can reverse the process so that the shield 30′ ends up back in the configuration shown in FIG. 27. Although not shown, it is possible to arranged or form another external thread similar to the thread 17^{VI} between the thread 17^{VI} and the projection 16^{VI} but close to and spaced from the projection 16^{VI}. This would allow the shield 30′ to be retained or locked in the fully extended position in a manner similar to it being locked in the retracted position as shown in FIG. 28.

In the drawings various features are shown as one-piece members or elements which are integrally formed. However, each one-piece or integrally formed member can also be made of multiple parts or elements. Materials for each of the various elements can be made medical grade synthetic resins or metals such as stainless steel. Also, the invention, illustratively disclosed herein, suitably may be practiced in the absence of any element which is not specifically disclosed herein and/or may utilize only those elements shown in one or more of the drawings.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A biopsy device comprising:
a body;
a cutting edge; and
at least one of:
a safety cover selectively lockable in at least one retracted position;
a safety cover selectively movable to at least one retracted position;
a safety cover axially retainable in at least one retracted position;
a safety cover axially lockable in at least one retracted position;
a movable safety cover;
a depth adjustment member; and
an arrangement for moving the cutting edge relative to a skin engaging surface.

2. The device of claim 1, wherein the body comprises the cutting edge.

3. The device of claim 1, wherein the device utilizes the depth adjustment member.

4. The device of claim 3, wherein the depth adjustment member is mounted to the body.

5. The device of claim 3, wherein the depth adjustment member is movably mounted to the body.

6. The device of claim 1, wherein the device utilizes the movable safety cover.

7. The device of claim 6, wherein the movable safety cover covers the cutting edge when in an initial position.

8. The device of claim 3, wherein the movable safety cover is movably mounted to a skin contacting end of the body.

9. The device of claim 1, wherein the device utilizes the depth adjustment member and the movable safety cover.

10. The device of claim 9, wherein movement of the movable safety cover is limited by the depth adjustment member.

11. The device of claim 1, wherein the device utilizes the movable safety cover and at least one spring for biasing the movable safety cover.

12. The device of claim 1, wherein the device further comprises a device for preventing reuse.

13. The device of claim 1, wherein the device further comprises a device movable from an initial position to a position preventing reuse of the device.

14. The device of claim 1, wherein the body comprises an elongated generally cylindrical body.

15. A biopsy device comprising:
a body;
a skin engaging surface;
a cutting edge; and
at least one of:
an arrangement for moving the cutting edge relative to the skin engaging surface;
an arrangement for moving the cutting edge at least axially toward the skin engaging surface;
an arrangement for causing rotation of at least one of the cutting edge and the body; and
an arrangement for allowing at least one of the cutting edge and the body to be coupled to a rotatable portion of a powered device.

16. The device of claim 15, wherein the skin engaging surface is arranged on a cover mounted to the body and the cutting edge is arranged on an axially moveable holder at least partially disposed in the body.

17. The device of claim 15, further comprising a trigger system and a device for moving the cutting edge to a trigger-set position.

18. The device of claim 15, further comprising a depth adjustment system.

19. A method making the device of claim 1, the method comprising:
arranging a safety shield on a body.

20. A single-use or disposable biopsy device comprising:
a cutting edge; and
a safety cover,
wherein at least one of:
the safety cover is movable relative to the cutting edge; and
the cutting edge is movable relative to the safety cover.
